(19) United States
(12) Patent Application Publication
(10) Pub. No.: US 2012/0172763 A1
(43) Pub. Date: Jul. 5, 2012

(54) MEASUREMENT DEVICE

(75) Inventors: Marcus James King, Christchurch (NZ); Lan Le-Ngoc, Christchurch (NZ); Julian Kyle Verkaaik, Christchurch (NZ)

(73) Assignee: Industrial Research Limited, Lower Hutt (NZ)

(21) Appl. No.: 13/380,145

(22) PCT Filed: Jul. 1, 2010
(86) PCT No.: PCT/NZ2010/000132

§ 371 Date: Mar. 7, 2012

(30) Foreign Application Priority Data
Jul. 1, 2009 (NZ) 578140

(51) Int. Cl. A61B 5/22; A61B 5/11

(52) U.S. Cl. 600/595

(57) ABSTRACT

A handheld measurement device (10) for enabling a user to measure a person's muscle strength and range of motion associated with a limb movement about a joint in a movement plane. The device comprises a contact surface (28) that is arranged to contact a part of the person's limb, a 3D orientation sensor that is arranged to sense the 3D orientation of the device in 3D space and generate representative 3D orientation signals during the limb movement, and a force sensor that is arranged to sense the force applied by the person's limb to the contact surface and generate representative force signals. A control system receives the 3D orientation and force signals and processes those signals to generate force data and angular rotation data.

### FIELD OF THE INVENTION

The present invention relates to a measurement device for use in patient rehabilitation applications, such as physiotherapy. In particular, although not exclusively, the measurement device can be used by a physiotherapist to assess a patient's strength and range of motion for various limb and joint movements.

### BACKGROUND TO THE INVENTION

Physiotherapists use various measurement devices and systems to assess a patient's ability, rate of recovery and the effectiveness of particular physiotherapy regimes. Muscle strength and range of motion assessments are most commonly used to assess a patient's progress during rehabilitation after injury or illness. The use of measurement tools and devices for assessing strength and range of motion of various limbs and associated joints can greatly assist a physiotherapist to accurately gauge a patient's progress and rate of recovery over time. The information from such assessments can then be used to gauge the effectiveness of any particular physiotherapy regime or exercises being carried out by the patient.

Measurement systems such as isokinetic dynamometers, are known for measuring a patient's muscle strength across a range of motion. These measurement systems typically require a subject to be strapped into a chair with a robotic arm driving their limb motion. In particular, often a torque sensor arm being driven in an arc by a variable speed motor is employed and the patient pushes against the arm through their range of motion. A pair of graphs is produced by the system that records torque and angle of the arm. Such measurement systems are typically used as research laboratory tools and are generally too large and expensive for using in a clinical environment for assessing patient rehabilitation.

Smaller hand-held measurement devices have been proposed that are more suitable for a clinical environment. For example, U.S. Pat. No. 6,729,801 describes a hand-held apparatus that is capable of selectively testing muscle strength in one mode and range of motion in another mode. Another hand-held measurement device, proposed in international PCT patent application publication WO 2006/038822, is capable of making isokinetic limb assessments of muscle strength over a range of motion by simultaneously sensing both force applied to the device by the limb and angular movement of the limb. These hand-held devices employ inclinometers to sense range of motion with respect to gravity.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

It is an object of the present invention to provide an improved handheld measurement device for measuring the muscle strength and range of motion associated with a person's limb movement about a joint, or to at least provide the public with a useful choice.

### SUMMARY OF THE INVENTION

In a first aspect, the present invention broadly consists in a handheld measurement device for enabling a user to measure a person's muscle strength and range of motion associated with a limb movement about a joint in a movement plane, comprising: a handheld housing having a contact surface that is arranged to contact a part of the person's limb during the limb movement; a 3D orientation sensor mounted within the housing that is arranged to sense the 3D orientation of the device in 3D space and generate representative 3D orientation signals during the limb movement; a force sensor associated with the contact surface that is arranged to sense the force applied by the person's limb to the contact surface and generate representative force signals during the limb movement; and a control system that is arranged to concurrently receive the 3D orientation signals and force signals from the respective sensors during a limb movement and process those signals to generate force data indicative of the force applied by the person's limb to contact surface during the limb movement and angular rotation data indicative of the angle of rotation of the limb about the joint in the movement plane during the limb movement.

Preferably, the control system may be arranged to process the 3D orientation signals from the 3D orientation sensor to generate 3D orientation representations of the device with reference to a 3-axis local device coordinate system and a 3-axis global coordinate system during the limb movement.

Preferably, the 3D orientation sensor comprises a 3-axis accelerometer that may be arranged to generate accelerometer signals representing the three orthogonal components of the gravity vector in the local device coordinate system and a 3-axis magnetometer that is arranged to generate magnetometer signals representing the three orthogonal components of the Earth's magnetic field vector in the local device coordinate system, and wherein the control system may be arranged to generate the 3D orientation representations based on the accelerometer and magnetometer signals.

Preferably, the control system may be arranged to generate the angular rotation data based on the orientation of a reference vector in the local device coordinate system.

Preferably, the control system may be arranged to extract the orientation of the reference vector from the 3D orientation representations of the device during the limb movement.

Preferably, the reference vector may be a vector substantially normal to the contact surface of the handheld housing.

Preferably, the angular rotation data represents the angular rotation of the reference vector in the movement plane and which corresponds to the angular rotation of the limb about its joint in the movement plane.

Preferably, the control system may be arranged to generate angular rotation data in the form of a single Range of Motion (ROM) angle representing the angle between the reference vector at the start and end of a limb movement based on a dot-product calculation of the start and end reference vectors.

Preferably, the control system may be further arranged to extract from the 3D orientation representations information indicative of the orientation of the movement plane for a limb movement relative to the 3-axis global coordinate system. More preferably, the movement plane may be defined as the plane extending between the reference vectors at the start and end positions of a limb movement.

In one form, control system may be arranged to output information indicative of whether the orientation of the movement plane corresponds to a substantially horizontal plane in the global coordinate system within a predefined tolerance range. Additionally, or alternatively, the control system may be arranged to output information indicative of whether the orientation of movement plane corresponds to a substantially vertical plane in the global coordinate system within a predetermined tolerance range.

Preferably, the control system may be arranged to generate a movement plane orientation angle representing the orientation of the movement plane relative to a reference plane.

Preferably, the control system may be arranged to generate information indicative of the orientation of the movement plane by determining the vector normal to the movement plane based on a cross-product calculation of the start and end reference vectors.

Preferably, the control system may be arranged to generate the angular rotation data representing the angular rotation of the limb about the joint relative to a preset anatomical joint reference axis. More preferably, the control system may be operable to extract the anatomical join reference axis from the 3D orientation representation of the device when the limb is in contact with the contact surface of the device and aligned with the desired anatomical joint reference axis. By way of example, the control system may further comprise a user interface that is operable by a user to set and store the anatomical joint reference axis prior to a limb movement measurement.

Preferably, the control system may be arranged to generate the 3D orientation representations of the device in the form of 3×3 rotation matrixes comprising values that represent the absolute orientation of this device in the global coordinate system.

Preferably, the control system may further comprise a user interface and may be arranged to receive input from a user via the user interface as to the start and end positions of a limb movement and wherein the control system is arranged to generate angular rotation data in the form of a ROM angle of the limb movement between the start and end positions.

Preferably, the control system may be arranged to generate the ROM angle based on the total angular rotation of a vector normal to the contact surface between the start and end positions of the limb movement in the movement plane.

Preferably, the control system may be arranged to generate force data and angular rotation data representing the force applied by the limb to the contact surface and the corresponding angular position of limb during the limb movement so as to generate measurement data indicative of muscle strength over the entire ROM of the limb movement.

Preferably, the control system may be arranged to generate force data comprising any one of the following: peak force, maximum force, or average force strength based on the force applied over the entire limb movement.

Preferably, the control system may further comprise a user interface to enable a user to operate the device.

In another form, the 3D orientation sensor may comprise one or more accelerometers and one or more gyroscopes that are together arranged to sense the 3D orientation of the device in 3D space and generate representative 3D orientation signals.

Preferably, the force sensor may comprise a load cell that is arranged to convert the force applied to the contact surface of the housing into a representative force signal.

Preferably, the housing may be grippable by a single hand of the user such that the contact surface of the housing can be held the user against a part of another person's limb during a limb movement.

Preferably, the movement plane may be any of the following: horizontal, vertical or arbitrary.

In a second aspect, the present invention broadly consists in a handheld sensor unit for enabling a user to measure a person's muscle strength and range of motion associated with a limb movement about a joint in a movement plane, comprising: a handheld housing having a contact surface that is arranged to contact a part of the person's limb during the limb movement; a 3D orientation sensor mounted within the housing that is arranged to sense the 3D orientation of the device in 3D space and generate representative 3D orientation signals during the limb movement; a force sensor associated with the contact surface that is arranged to sense the force applied by the person's limb to the contact surface and generate representative force signals; and a control system that is arranged to concurrently receive the 3D orientation signals and force signals from the respective sensors during a limb movement and transmit those to an external device.

Preferably, the control system may comprise a communication module that is arranged to transmit the 3D orientation signals and force signals to an external device. In one form, the communication module may be configured for wired connection and transmission of data with an external device. In another form, the communication module may be configured for wireless communication of data with an external device.

Preferably, the control system may further comprise a user interface to enable a user to operate the sensor unit to begin sensing at the start position of the limb movement and halt sensing at the end position of the limb movement.

The sensor unit may further have any one or more features outlined in respect of the measurement device of the first aspect of the invention.

In a third aspect, the present invention broadly consists in a method of measuring a person's muscle strength and range of motion associated with a limb movement about a joint, comprising the steps of: (a) applying the contact surface of a handheld measurement device or sensor unit of either of claim 1 or claim 28 to a part of the person's limb with resistance; (b) causing the person to move their limb through its full range of motion about the joint in a movement plane; (c) measuring the force signals and 3D orientation signals from the sensors of the device or unit during the limb movement; and (d) processing the force signals and 3D orientation signals to generate output data representing the person's muscle strength over their range of motion for the limb movement.

Preferably, step (d) may comprise generating 3D orientation representations of the device or unit with reference to a 3-axis local device coordinate system and a 3-axis global coordinate system based on the 3D orientation signals. More preferably, step (d) may comprise generating the 3D orientation representations of the device or unit in the form of rotation matrices that represent the absolute orientation of the device or unit in the global coordinate system.

Preferably, step (d) may further comprise processing the series of rotation matrices to generate angular rotation data representing the angle of rotation of the limb about the joint based on the rotation of a reference vector in the local device coordinate system.

More preferably, the reference vector may be substantially normal to the contact surface of the device or unit.

Preferably, step (d) may comprise generating a measurement of range of motion of the limb based on the total angle of rotation of the reference vector in the movement plane between the start and end positions of the limb movement.

Preferably, the method may further comprise the step of setting an anatomical joint reference axis prior to starting the limb movement by aligning the person's limb within the desired anatomical joint reference axis and operating the device or unit to extract and store the anatomical joint reference axis based on the 3D orientation signals sensed at that position; and wherein angular rotation data representing the angle of rotation of the limb about the joint is generated relative to the stored anatomical joint reference axis.

The output data may be presented in the form of numerical outputs, such as a numerical force data output representing, for example, the maximum force applied during the limb movement, and a numerical angle data output representing, for example, the range of motion of the limb movement, such as the total 2D angular rotation of the limb in the movement plane during the limb movement. Additionally or alternatively, the output data may be presented graphically, such as a graph of force against 2D angular rotation of the limb in the movement plane during the limb movement.

In a fourth aspect, the present invention may broadly consist in a method of generating a measurement of the angular rotation of a person's limb about a joint in a movement plane based on signals received from a 3D orientation sensor, having an accelerometer and magnetometer, and which is coupled to move with the limb during movement, the method comprising the steps of: (a) defining a 3-axis local coordinate system for the sensor and a 3-axis global coordinate system; (b) receiving accelerometer and magnetometer signals during the limb movement; (c) generating rotation matrices representing the absolute 3D orientation of the sensor with reference to the 3-axis local coordinate system and 3-axis global coordinate system; (d) processing the rotation matrices to extract angular rotation data relating to the angular rotation of a reference vector of the 3-axis local coordinate system in the movement plane of the 3-axis global coordinate system; and (e) generating a measurement of angular rotation of the limb based on the angular rotation data.

Preferably, step (e) may comprise generating a measurement of the total angular rotation of the limb during the limb movement.

Preferably, step (e) may comprise generating a measurement of the angular rotation of the limb with reference to an anatomical joint reference axis. More preferably, the method may further comprise the step of setting an anatomical joint reference axis prior to limb movement.

The term “comprising” as used in this specification and claims means “consisting at least in part of”. When interpreting each statement in this specification and claims that includes the term “comprising”, features other than that or those prefaced by the term may also be present. Related terms such as “comprise” and “comprises” are to be interpreted in the same manner.

The invention consists in the foregoing and also envisages constructions of which the following gives examples only.

### BRIEF DESCRIPTION OF THE DRAWINGS

Preferred embodiments of the invention will be described by way of example only and with reference to the drawings, in which:

FIG. 1a shows a top-side perspective view of a first preferred form of measurement device of the present invention from the front end;

FIG. 1b shows a right-side view of the measurement device of FIG. 1a;

FIG. 2a shows an under-side perspective view of the measurement device of FIG. 1a;

FIG. 2b shows a left-side perspective view of the measurement device of FIG. 1a;

FIG. 3 shows an under-side view of the first preferred form measurement device, and in particular shows the contact pad and display;

FIG. 4a shows a perspective cross-sectional view of an alternative housing for the first preferred form measurement device, and shows the arrangement of the main components;

FIG. 4b shows a partially exploded perspective view of the housing and components of the measurement device of FIG. 4a;

FIG. 5 shows a perspective view of a preferred form force sensor of the measurement device of FIGS. 4a and 4b;

FIG. 6 shows a high-level schematic block diagram of the main modules of the first preferred form measurement device, and optional communication modules;

FIG. 7a shows a schematic representation of the 3D axes of the local and global coordinate systems that form the reference frames for the 3D orientation sensing capability of the first preferred form measurement device;

FIG. 7b shows 3D axes of the global coordinate system with reference to the gravity vector and Earth's magnetic field vector;

FIG. 8 shows the first preferred form measurement device in use with a therapist holding the device against the patient's forearm while they perform a right elbow flexion;

FIGS. 9a and 9b show an example of a patient limb movement in which patient's range of motion is in the vertical plane with a right elbow flexion;

FIGS. 9c and 9d show an example of a patient limb movement in which the patient's range of motion is in the horizontal plane with a right shoulder joint rotation;

FIGS. 9e and 9f show an example of a patient limb movement in which the patient's range of motion is in an arbitrary plane with a right shoulder rotation through both the vertical and horizontal planes;

FIG. 10 shows a graph generated from the data measured by the first preferred form measurement device, and in particular shows force (muscle strength) versus angle (range of motion) for a particular patient limb movement;

FIGS. 11a and 11b show top-side perspective views from various angles of a second preferred form measurement device of the present invention from the front end;

FIG. 11c shows an under-side perspective view of the measurement device of FIGS. 11a and 11b;

FIG. 11d shows a top-side perspective view of the measurement device of FIGS. 11a and 11b from the back end;

FIG. 12 shows a high-level schematic block diagram of the main modules of the second preferred form measurement device;

FIG. 13 shows a screenshot of the user interface of a preferred database management system associated with the measurement device of the present invention;

FIG. 14 shows a screenshot of a preferred graphing module of the database management system;

FIG. 15 shows a screenshot of a preferred patient data module of the database management system;

FIG. 16 shows a screenshot of a preferred data transfer module for transferring data between the measurement device and database management system; and

FIG. 17 shows a screenshot of an enlarged graph generated by the graphing module of the database management system.

### DETAILED DESCRIPTION OF PREFERRED FORMS

### Overview

The present invention relates to a handheld portable measurement device or instrument for measuring the range-of-motion (ROM) and strength associated with a person's limb movements about various joints in the human body. The measurement device primarily designed for clinicians and therapists to use in a patient rehabilitation environment in which it is necessary to periodically assess a patient's ability and progress with respect to a rehabilitation plan. However, it will be appreciated that the measurement device could be employed in any application in which a measurement of muscle strength and range of motion for human body joints is required.

In operation, the measurement device is held by the clinician against a part of the patient's limb that is associated with the joint under assessment. The patient is then instructed to move their limb and the device makes the required measurements as will be explained in further detail later. The measurement device is capable of simultaneously and continuously measuring both muscle strength (force) and ROM for a patient's limb movement about a joint in 3D space.

In brief, the measurement device comprises a 3D orientation sensor that is capable of continuously sensing the 3D orientation of the device in 3D space and the 3D orientation information can be converted into a ROM representation for the patient's limb movement about the joint in a movement plane. The measurement device also includes a force sensor that is arranged to continuously sense the force applied to the measurement device by the patient's limb during the limb movement and generates force data representing the muscle strength associated with the limb movement about the joint.

The measurement device is capable of measuring the ROM and muscle strength associated with limb movements about any suitable joints, including hinge-type joints such as knees and elbows, and also ball-type joints such as hips, and more complex joints such as shoulders and ankles. The limb movement can be carried out in any anatomical plane, whether horizontal, vertical or an arbitrary movement plane having both horizontal and vertical components.

### First Preferred Form—Handheld Measurement Device

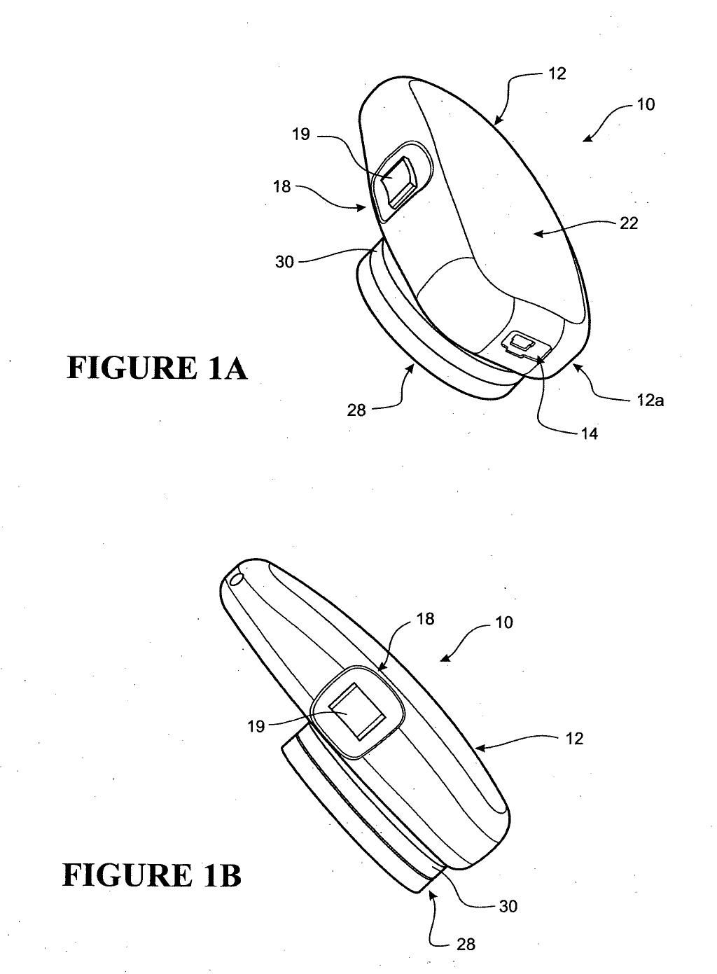

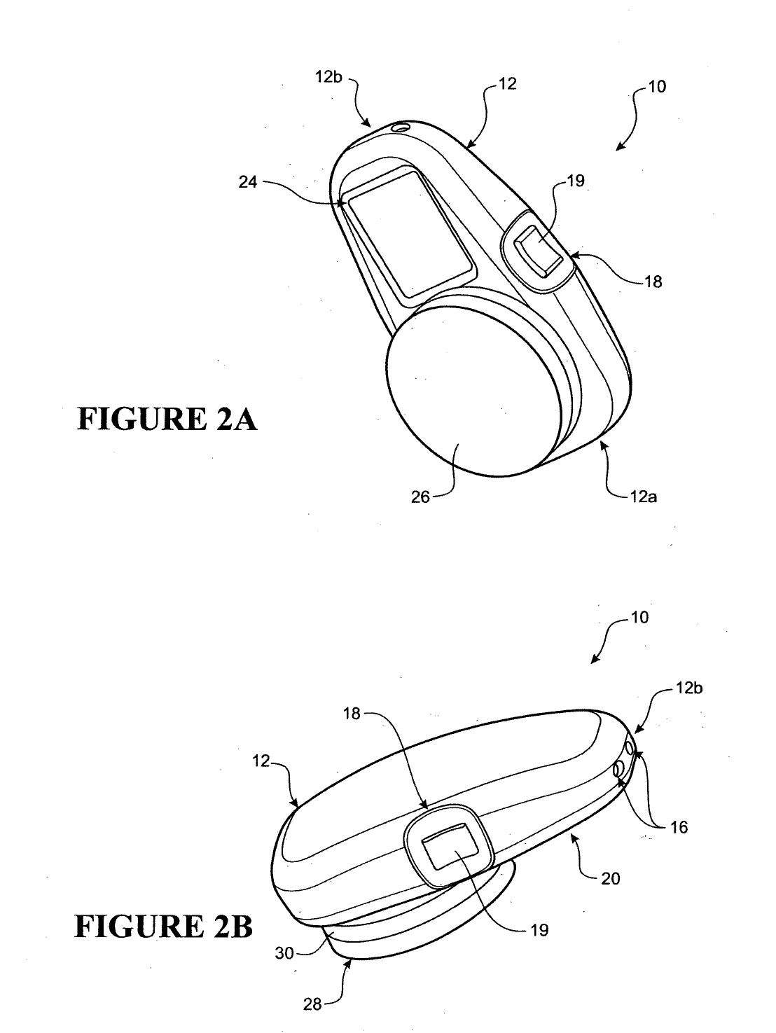

Referring to FIGS. 1a-2b, the first preferred form of the handheld measurement device 10 comprises a housing 12 that is preferably ergonomic in shape and able to be held by a single hand of a user. In the first preferred form, as shown in FIG. 1a, the front end 12a of the housing 12 is provided with an external device interface socket 14, such as a universal serial bus (USB) port or the like, for communicating with other external devices and transferring data. The external device may be a Personal Computer for example, whether a laptop, desktop, Personal Digital Assistance (PDA) or other computing device, portable or otherwise. With reference to FIG. 2b, the back end 12b of the housing 12 is preferably tapered and provided with one or more indicator Light Emitting Diodes (LEDs) 16 that signal the operational status of the device.

Finger recesses 18 to enable a user to grip and hold the device with, for example, their thumb and index finger are preferably provided toward the middle of the left and right sides of the housing. In particular, a user can grip the opposed finger recesses 18 with a thumb and index finger respectively, and their remaining fingers can wrap underneath the bottom face 20 of the back end 12b of the housing 12. In the first preferred form, the top face 22 of the housing is preferably smooth. On the opposite side, the bottom face 20 of the housing 12 is preferably provided with an output display screen 24, such as a Liquid Crystal Display (LCD) 24, at or toward the back end 12b of the housing and a contact surface 26 at or toward the top end 12a of the housing.

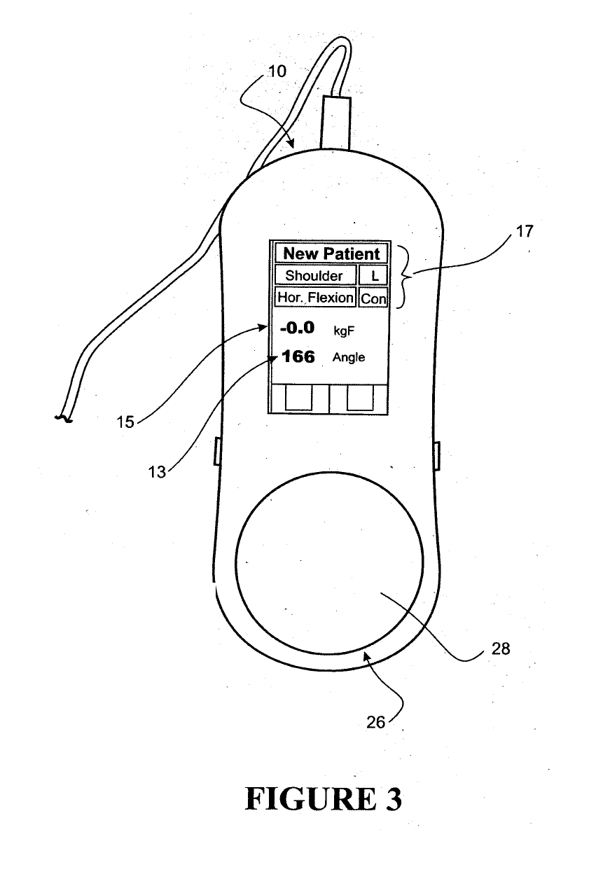

With reference to FIG. 3, the LCD 24 may be arranged to display various forms of the data measured by the device during and after a limb movement. By way of example only, the LCD 24 may be arranged to display a continuous measurement of the ROM 13 of the limb through any arbitrary plane of movement with reference to a user set anatomical reference axis (or zeroed axis) in that plane, and in particular the ROM may be represented as a single angle in degrees. Additionally, the LCD 24 may be arranged to concurrently display a continuous measurement of the force 15 applied by the limb to the contact surface 26 of the measurement device, and this may be represented in units such as kilogram-force (kgF or kg), Newtons (N), Pounds force (lb) or in any other metric or imperial unit representing force. The output display may also be configured to show information indicative of the movement plane through which the limb moved, patient data, the joint being assessed, and the limb movement being performed. The patient data 17 can include any relevant patient information, such as the patient's name, date of birth, joint and movement description, and other relevant identification or other details.

It will be appreciated that the control electronics, including the sensor interface circuitry, power supply circuitry, external device interface circuitry are provided on a circuit board mounted within the housing 12 of the measurement device 10. In the first preferred form, the measurement device is powered by an onboard battery supply, which may be rechargeable. The control system of the measurement device will be explained in more detail later.

### Force Sensor

With reference to FIGS. 1a-3 the contact surface 26 of the measurement device is arranged to bear against a portion or bony protrusion of the patient's limb as the clinician holds the measurement device 10 against the limb during an instructed movement. The contact surface 26 may be associated with the force sensor or an integral part of the force sensor. In the first preferred form, the force sensor comprises a contact pad or plate 28 and a force transducer 30 mounted to the housing 12.

The contact pad 28 provides the contact surface 26 and may be permanently or releasably attached to the force transducer 30. In the first preferred form, the contact pad 28 is permanently mounted to the force transducer 30 by adhesive or any other form of suitable mounting system. In alternative forms, a releasable coupling system may be provided for mounting the contact pad 28 to the force transducer 30, such as a magnetic coupling system or the like. In such forms, different shapes or sizes of contact pads 28 could be attached to the measurement device to customise it for particular limb assessments or patients.

In the first preferred form, the contact pad 28 protrudes from the bottom face 20 of the housing 12. The contact pad 28 provides a shaped and padded compression surface for the abutting against a part of the patient's limb or other suitable body part associated with the joint under assessment. It will be appreciated that the contact pad 28 is not necessarily essential to the measurement device and in alternative forms the force transducer 30 itself may form the contact surface 26. Additionally, the contact surface 26 need not necessarily be displaced from the bottom face 20 of the housing and could alternatively be flush with the bottom face if desired.

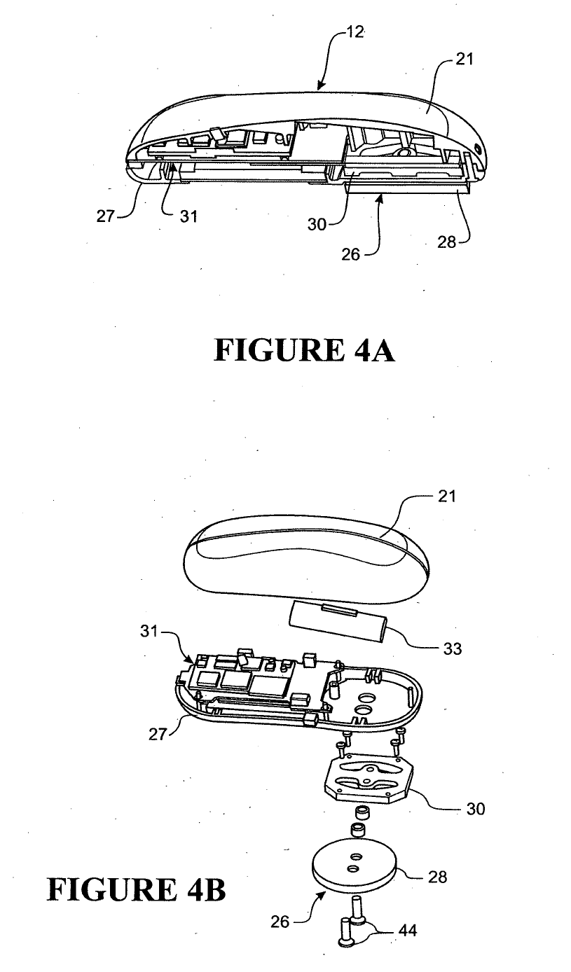

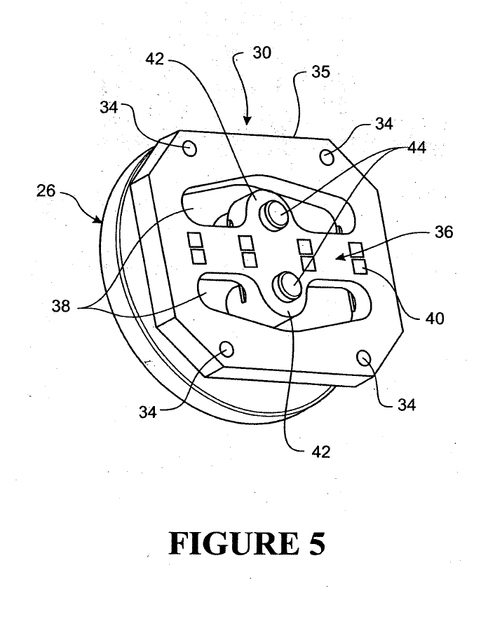

FIGS. 4a, 4b and 5 show an alternative form of housing for the first preferred form measurement device relative to the housing shown in FIGS. 1a-3. By way of example only, a preferred form arrangement of the main components of the measurement device and a preferred form configuration of the force sensor will be described with reference to FIGS. 4a, 4b and 5, with like numerals representing like components.

Referring to FIGS. 4a and 4b, the housing 12 comprises an upper casing part 21 that securely couples to a lower casing part 27 to form an enclosed casing. A Printed Circuit Board (PCB) 31 is mounted within the housing and this provides the control system electronics and 3D orientation sensor components. A battery 33 is also mounted securely within the casing. The force transducer 30 and its associated contact pad 28 are securely screw-mounted to the lower casing part 27, and their configuration will be explained further below.

The force sensor may comprise any form of force transducer, load cell, strain gauge or device that can convert, directly or indirectly, force applied by or between the patient's limb and contact surface 26 of the measurement device. Further, it will be appreciated that a pressure transducer could be employed in alternative forms of the device to indirectly measure applied force via sensing pressure applied to the contact surface 26 and then converting that into representative force data.

With reference to FIG. 5, the preferred form force transducer 30 comprises a base plate 35 having mounting apertures 34 for screw-mounting the transducer to the lower casing part 27. The base plate 35 comprises a central beam portion 36 that is formed in between two elongate apertures 38 that extend through the base plate. A series of strain gauge resistors 40 are provided on the central beam portion 36. In the preferred form, there are eight foil-type strain gauge resistors fixed across the upper surface of the central beam portion 36, and these are coupled in a Wheatstone bridge to create a load cell. The central beam portion 36 comprises two integral side lugs 42, each extending outwardly from a side of the central beam portion. In the preferred form, the side lugs 42 are located at or toward the center of the central beam portion 36. Each side lug 42 comprises a threaded mounting aperture and mounting screws 44 extend through the contact pad 28 and into the mounting apertures to secure the contact pad to the base plate 35 of the force transducer 30. This arrangement ensures that the central beam portion 36 supports the load when a patient pushes against the contact pad 28 and the clinician holds the device 10 in a manner that will be described later. It will be appreciated that any other form of mounting mechanism or system could alternatively be utilised to secure the contact pad 28 permanently, or releasably, to the force transducer 30.

The central beam portion 36 of the base plate is shaped, sized and/or formed with material that enables it to deform or flex slightly under loading, such as the force applied against the contact pad 28 by a patient. In operation, the central beam portion 36 deforms slightly under the designed loadings and this deformation may be measured by the strain gauge resistors 40 of the load cell as described below.

The strain gauges resistors 40 of the force transducer 30 are connected with wiring to the control circuitry of the control system and arranged to generate force signals representing the force applied between the patient's limb and the contact pad 28. These force signals provide a measure of the muscle strength associated with the limb as it moves about the joint under assessment. The force transducer 30 is arranged to continuously sense applied force and transmit representative force signals to the control electronics for subsequent storage, processing, transfer and/or display.

### 3D Orientation Sensor

A 3D orientation sensor is mounted within the housing and is arranged to generate 3D orientation signals representing the 3D orientation of the measurement device. In the preferred form, the 3D orientation sensor comprises an accelerometer and a magnetometer. The accelerometer measures force due to gravity or a change in velocity and outputs representative voltage signals. The accelerometer is preferably a 3-axis accelerometer that effectively measures the angular orientation or rotation of the measurement device with respect to gravity in the vertical plane when the device is held in any plane. The magnetometer measures the direction of the Earth's magnetic field and outputs representative voltage signals. The magnetometer is preferably a 3-axis magnetometer that is arranged to measure the angular orientation or rotation of the measurement device with respect to the Earth's magnetic field in the horizontal plane when the device is held in any plane.

The signals sensed by the accelerometer and magnetometer of the 3D orientation sensor are processed to provide 3D orientation data or information representing the 3D orientation of the measurement device in 3D space at any point in time. This 3D orientation data is continuously sensed during the limb movement and can be processed to extract various angular movement data relating to the movement of the measurement device. By way of example, the angular rotation of the vector normal to the contact surface 26 of the device can be determined in any arbitrary movement plane, such as a horizontal plane, vertical plane or an arbitrary plane having a combination of vertical and horizontal components. The angular rotation of the vector normal can be used to represent the ROM of the limb movement in the movement plane.

In an alternative form, it will be appreciated that the 3D orientation sensor may comprise one or more accelerometers and one or more gyroscopes that are together arranged to sense the 3D orientation of the device in 3D space and generate representative 3D orientation signals.

### Control System

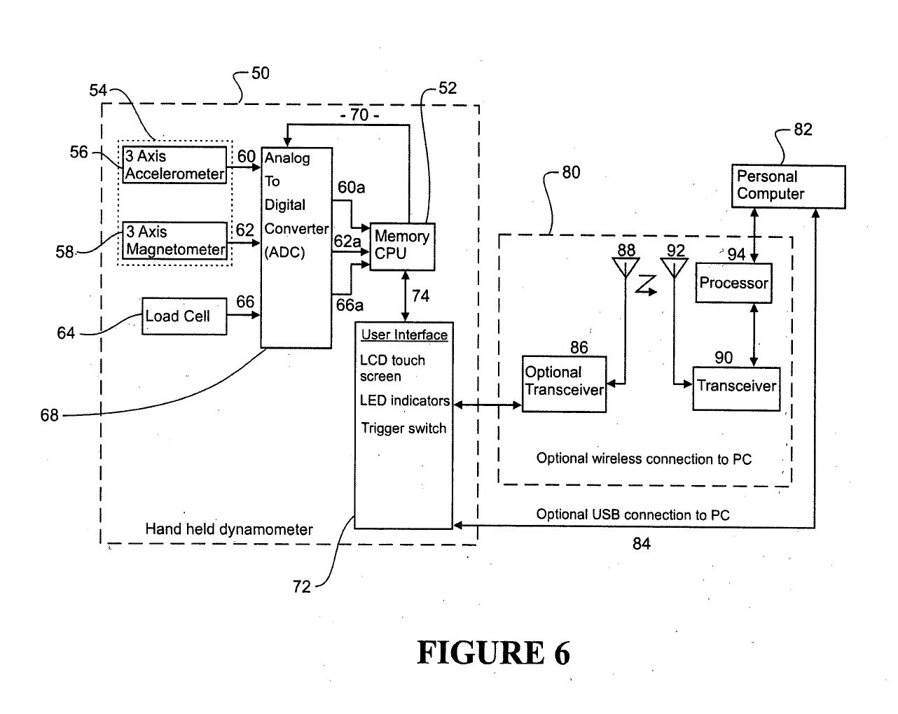

It will be appreciated that various control system configurations could be employed depending on the device requirements. By way of example and with reference to FIG. 6, the main modules of the control system 50 of the first preferred form measurement device will now be explained. In brief, the control system mainly operates the 3D orientation sensor and force sensor, and simultaneously receives the 3D orientation signals and force signals sensed during a limb movement. In the preferred form, the control system is triggered to operate by operation of switch(es) 19.

The control system 50 comprises a main controller 52. The main controller 52 may be a programmable microprocessor such as a microcontroller, digital signal processor, or any other suitable type of programmable or computing device. The main controller may have onboard memory and/or may be connected to an external memory module or modules for storing measured data.

As previously mentioned, the 3D orientation sensor 54 of the measurement device comprises a 3-axis accelerometer 56 and a 3-axis magnetometer 58, each of which generates respective voltage signals 60,62 that represent the 3D orientation of the measurement device. The force sensor 64 of the measurement device generates a voltage signal 66 representing the force applied to the measurement device. The analogue voltage signals 60,62,66 generated by the sensors are preferably converted to respective digital signals 60a,62a,66a by an analogue-to-digital converter (ADC) 68. The ADC module 68 is controlled by a control signal 70 form the main controller 52. In particular, the control signal 70 generated may be the ADC's sampling clock.

In operation, the main controller 52 is arranged to simultaneously receive the digital data signals 60a,62a,66a from the 3D orientation sensor and force sensor for processing, storage and/or outputting. In particular, the measured data signals can be processed to generate representative output data about the ROM and muscle strength associated with a patient's limb movement. The operation of a measurement device and processing of the 3D angle and force signals will be described in more detail later.

### User and External Device Interface

In the first preferred form, the main controller 52 has an associated user interface module 72 that is operable by a user to control and configure the measurement device and its settings during a limb movement assessment. The user interface module 72 can also be operated to access, display, transfer or store data measured by the device. It will be appreciated that the main controller 52 and the user interface module 72 are operatively connected and communicate via various control signals 74.

In the first preferred form, the measurement device comprises a user interface 72 having an operable trigger or switch 69 that allows a user to start and stop the measurement recordings of the device in operation. Preferably, the switch 19 is provided in the finger recesses 18 as shown and described with reference to FIGS. 1a-2b.

The preferred form user interface 72 also comprises an output display 24 which is arranged to display the measurement readings in numerical, graphical or any other appropriate format. Preferably, but optionally, the output display may include touch screen interface capability to enable the user to select measurement modes, configure various device settings, and access data for display. For example, the user can use the touch screen to scroll through stored data, display selected data, compare measured data, export or import data and the like. The output display may, for example, be an LCD touch screen. Alternatively, a menu may be scrolled through by navigation buttons that are separate from the LCD screen.

Optionally, the measurement device may comprise an LED array 16 that is operable to signal the device status. In addition, an operable audible output device may be provided, such as a piezoelectric transducer. The control system may activate the audible output device to output one or more feedback, status or warning sounds indicative of various aspects of a measurement assessment, for example but not limited to the start and stop times of a measurement, whether the calculated orientation of the movement plane corresponds to a user selected movement plane, e.g. horizontal or vertical planes within desired tolerances, or any other aspect.

The control system 50 of the measurement device 10 may optionally have an associated external device interface module that is arranged to transfer measured data to external devices, such as a Personal Computer 82, database system, external memory or the like. For example, in a clinical environment the clinician may make a limb assessment using the handheld measurement device and then upload or transfer the measured data to their Personal Computer or patient database system for storage, future analysis, and/or comparison with future or previous records or with a representative population database. The external device interface module may be a separate module or may be integrated with the user interface module 72. The external device interface module may employ bi-directional interface circuitry such that it can both transmit data to other devices and receive data from other devices.

In the first preferred form, the external device interface may be in the form of a hardwired connection, wireless connection, or both. By way of example, an external device interface socket 14, such as a USB connection and interface circuitry, may be provided for connecting to a Personal Computer 82 by a USB cable for uploading and downloading data, and for power supply charging. It will be appreciated that other hardwired interface cables and circuitry could alternatively be used, whether serial or parallel connection cables. Alternatively, or additionally, a wireless communication connection 80 may be provided for connecting wirelessly to a Personal Computer 82. For example, the measurement device may be provided with a transceiver 86 and aerial 88 for communicating over a wireless medium with a corresponding transceiver 90 and aerial 92 that are hardwired to the Personal Computer via a communication controller 94. It will be appreciated that the wireless communication connection may utilise any suitable communication protocol, including WiFi, Bluetooth or the like. Infrared communication is also possible in alternative forms of the measurement device.

As will be explained later, the user can operate the measurement device to deal with the measured data in various ways, including storing the measured data on the measurement device, displaying the data graphically or otherwise on the LCD 24, transferring the measured data to an external device, and/or processing the measured data as desired to generate meaningful clinical output values for assessment. It will be appreciated that the main controller can be arranged to process the measured data in real-time as the data is received or alternatively temporarily store the data for post-processing after the limb assessment has been completed.

### 3D Orientation Sensing—Processing Algorithms

In the first preferred form measurement device, the control system is arranged to process the 3D orientation signals from the 3D orientation sensor to generate angular rotation data representing the angle of rotation of the limb about the body joint under test in any arbitrary movement plane, whether horizontal, vertical or otherwise. In effect, the angular rotation data represents the 2D planar angle of rotation or 2D planar angular position of the limb in the movement plane, and is derived from continuous sensing of the 3D orientation of the measurement device. The control system may also be arranged to process the angular rotation data to generate a measurement of range of motion (ROM) of the limb movement about the joint in the movement plane. Optionally, the control system may further be configured to extract from the angular rotation data information indicative of the orientation of the movement plane in 3D space through which the limb was moved during the test.

In the first preferred form, the processing of the 3D orientation signals is performed onboard the measurement device by the main controller 52 of the control system that implements various processing algorithms for representing the real-time 3D orientation of the measurement device and for calculating the ROM of the device during a limb movement, and optionally the information indicative of the orientation of the movement plane through which the limb was moved during the test.

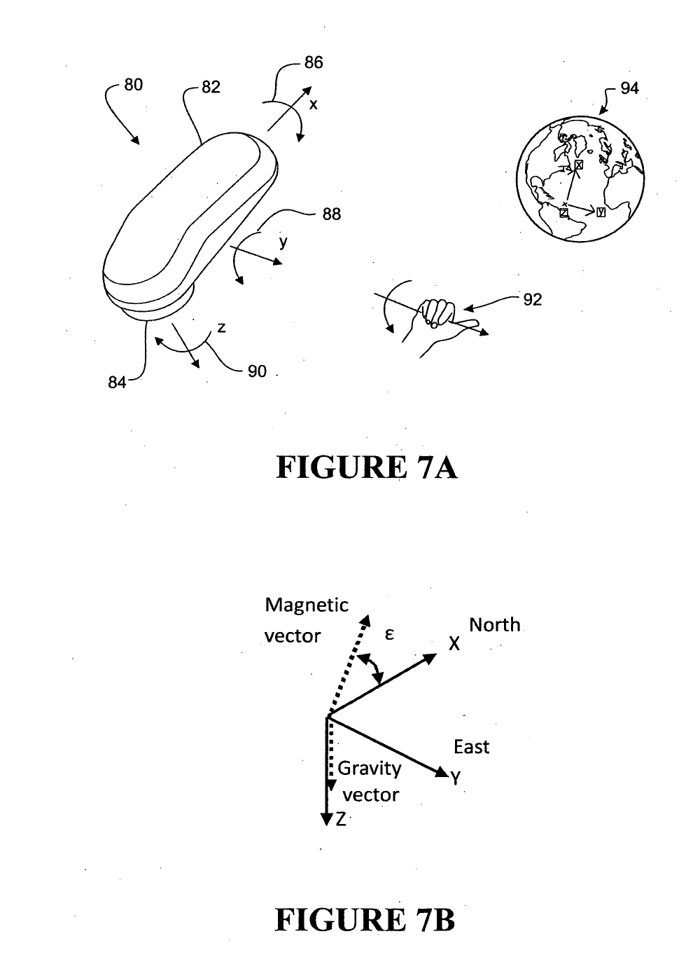

The 3D orientation representation of the device is based on 3-axis reference frames, including a measurement device local coordinate system and a global coordinate system. Referring to FIG. 7a, the 3-axis local measurement device coordinate system will be referred to as x,y,z and the 3-axis global coordinate system will be referred to as X,Y,Z. A schematic representation of the measurement device is indicated generally at 80 and comprises the handheld housing 82 and contact pad 84 carrying the contact surface.

By way of example, the local device coordinate system of the measurement device is defined with the x-direction being the longitudinal direction of the measurement device 80, the y-direction being the transverse direction, and the z-direction being the direction normal to the contact pad 84. The three directions or axes are preferably orthogonal to each other. The positive rotation directions show as arrows 86, 88, 90 for the respective x,y,z directions follow the right-handed rule as shown in schematic 92 such that the positive rotation occurs in the direction of the fingers. The definition of the global coordinate system X,Y,Z is shown schematically in the world coordinates of 94 with the X and Y directions being the longitudinal and transverse directions respectively in a plane and the Z-direction being in the normal direction into the plane. Again, the three axes or directions in the global coordinate system are orthogonal to each other.

As mentioned, the 3D orientation sensor comprises a 3-axis accelerometer and a 3-axis magnetometer mounted inside the housing and the signals generated by these components can be extracted and processed to identify the 3D orientation of the measurement device, and more importantly the rotation of the measurement device in a movement plane during a limb movement. The 3-axis accelerometer generates three voltage signals or readings with respect to the local coordinate system directions x,y,z and these accelerometer output signals will be referred to as Aₓ, A_{y}, A_{z} by way of explanation. Likewise, the 3-axis magnetometer generates three voltage signals or readings will be referred to as Mₓ, M_{y}, M_{z}. It will be appreciated that the x,y,z subscripts for the accelerometer and magnetometer signals represent the respective x,y,z directions in the measurement device local coordinate system.

For the purpose of explanation, assume that both the accelerometer and the magnetometer are calibrated and scaled such that:—

\sqrt{Aₓ²+A_{y}²+A_{z}²}=1

\sqrt{Mₓ²M_{y}²+M_{z}²}=1 (1)

Then the physical meanings of Aₓ, A_{y}, A_{z} are the three orthogonal components of the gravity vector in the local device coordinate system. The gravity vector in the global coordinate system is {0, 0, 1}^{T}, that is, pointing downward in the Z direction. The superscript T denotes the transpose of the vector and represents the column vector as a row vector transpose. Similarly, the physical meanings of Mₓ, M_{y}, M_{z} are the three orthogonal components of the Earth's magnetic vector in the local device coordinate system. The Earth's magnetic vector is dependent on the latitude location of where the device is being used, as well as other interference from iron objects in the vicinity. We can assume that the Earth's magnetic field is uniform within the operational space of the device, even if this is distorted by local metallic objects such as building super-structure. By way of example, in the Southern hemisphere the global coordinates of the magnetic vector is {cos ε, 0, −sin ε}^{T}, where ε is the inclination angle of the magnetic field. For example, in Christchurch, New Zealand, ε is approximately 60°. FIG. 7b shows the global coordinate system X,Y,Z and the gravity vector and Earth magnetic vector by way of example. It will be appreciated that the device processing algorithms can be calibrated for different locations in regard to the Earth's magnetic vector inclination angle.

In the first preferred form, the 3D orientation of the measurement device with respect to a fixed global coordinate system is represented by a 3×3 rotation matrix. More particularly, a 3D orientation representation algorithm is arranged to form the 3×3 rotation matrix from the 3-axis signals from the accelerometer and magnetometer, and the 9 components of the rotation matrix can be manipulated and processed to extract the required measurement device 3D orientation information, including the ROM during a limb movement and information indicative of the orientation of the movement plane in 3D space. For example, when the user moves the measurement device from one orientation to another during a patient's limb movement, the device angle of rotation (representing the patient's ROM) can be calculated from two rotation matrices, one which represents the 3D orientation of the device at the start of the limb movement and the other representing the device's 3D orientation at the end of the limb movement. Additionally, information indicative of the orientation of the movement plane through which the limb moved may be extracted based on the start and end 3D orientation of the measurement device.

### 3D Orientation Representation Algorithm

In the first preferred form, the main controller 52 is arranged to implement a 3D orientation representation algorithm that continuously processes the signals from the accelerometer and magnetometer, and generates 3×3 rotation matrices representing the 3D orientation of the device in real-time. The sampling rate of the accelerometer and magnetometer signals and rate of creation of the rotation matrices may be adapted to suit accuracy and design requirements. The rotation matrices stored during a limb movement are preferably stored for further processing, such as by a ROM calculation algorithm that will be explained in more detail later.

The 3D orientation representation algorithm will now be described. To generate a rotation matrix that defines the absolute orientation of the device with respect to the global coordinate system, three orthogonal vectors x, y, z, are needed. The gravity vector is in line with the global Z axis so that the vector {Aₓ₁, A_{y}, A_{z}}^{T} forms the Z vector automatically. The remaining two vectors can be found from the cross-product operations given in equations (2) and (3) as explained further below. The Earth's magnetic vector is not orthogonal to the gravity vector, however, a cross product between the gravity vector and the magnetic vector, {Mₓ, M_{y}, M_{Z}}^{T}, will give the eastward orthogonal vector {Eₓ, E_{y}, E_{Z}}^{T} as follows:

$$\{ \begin{matrix}{E}_{x} \\ {E}_{y} \\ {E}_{z}\end{matrix} \} = \{ \begin{matrix}{A}_{x} \\ {A}_{y} \\ {A}_{z}\end{matrix} \} \times \{ \begin{matrix}{M}_{x} \\ {M}_{y} \\ {M}_{z}\end{matrix} \} \qquad ( 2 )$$

The third orthogonal vector, the northward vector can be found as follows:

$$\{ \begin{matrix}{N}_{x} \\ {N}_{y} \\ {N}_{z}\end{matrix} \} = \{ \begin{matrix}{E}_{x} \\ {E}_{y} \\ {E}_{z}\end{matrix} \} \times \{ \begin{matrix}{A}_{x} \\ {A}_{y} \\ {A}_{z}\end{matrix} \} \qquad ( 3 )$$

The rotation matrix, R, to completely define the 3D orientation of the device is:

$$R = [ \begin{matrix}{N}_{x} & {E}_{x} & {A}_{x} \\ {N}_{y} & {E}_{y} & {A}_{y} \\ {N}_{z} & {E}_{z} & {A}_{z}\end{matrix} ] \qquad ( 4 )$$

This rotation matrix, R, can be utilised to transform any vector from the global coordinates into the local coordinates of the device. The inverse is the transformation from local coordinates into global coordinates. Since R is a normalised rotation matrix (det(R)=1), the inverse is simply the transpose of R, that is:

$${R}^{- 1} = {R}^{T} = [ \begin{matrix}{N}_{x} & {N}_{y} & {N}_{z} \\ {E}_{x} & {E}_{y} & {E}_{z} \\ {A}_{x} & {A}_{y} & {A}_{z}\end{matrix} ] \qquad ( 5 )$$

### ROM Calculation Algorithm

In the first preferred form, the ROM calculation algorithm is based on rotation of the vector substantially normal to the contact pad 84 associated with the force sensor of the device 80. The vector perpendicular or normal to the contact pad 84 is the z-axis of the local device coordinate system, and in the global coordinate system frame of reference, it is the third column of R^{T} as follows:

$${V}_{z} = \{ \begin{matrix}{N}_{z} \\ {E}_{z} \\ {A}_{z}\end{matrix} \} \qquad ( 6 )$$

The ROM measurement of a limb movement is the angle of rotation of the limb in a movement plane from one position (denotes position 1) to another (position 2) about the joint being tested. With reference to FIG. 8, in operation the measurement device 10 is placed with the contact pad 28 against the patient's limb 23 so that the clinician 25 can provide resistance to the motion of the limb and the patient is instructed to apply as much force as possible to move the limb in the direction of interest. In FIG. 8, the joint being assessed is the patient's right elbow and the contact pad 28 of the measurement device is held by the clinician 28 against the patient's inner forearm 23.

Then the angle of rotation of the z-axis (vector normal to the contact pad 28) of the measurement device from position 1 to position 2 is the same as the angle of rotation of the limb or ROM. This angle, θ, can be found from the dot product of V_{z1} and V_{z2} as follows:

cos θ=V_{z1}·V_{z2}=N_{z1}N_{z2}+E_{z1}E_{z2}+A_{z1}A_{z2} (7)

In general, the ROM angle θ will be conditioned to produce a positive angle between 0° and 359°. Generally, positions 1 and 2 will be selected to be the start and end positions of the limb movement so as to produce a ROM angle that corresponds to the total ROM of the limb movement, but it will be appreciated that equation (7) may be used to calculate the angle between any two other selected positions within the limb movement if desired.

While the preferred form algorithm for calculating the angular rotation of the limb about the joint is based on the angular rotation of the vector substantially normal to the contact surface, it will be appreciated that the rotation of other vectors in the local device coordinate system could alternatively be used to provide a measure of the rotation of the limb in other forms of the algorithm.

### Orientation of Movement Plane Calculation Algorithm

The ROM calculation algorithm, and in particular the final equation (7) above, calculates the angle between the vector normal to the contact pad of the measurement device at a first position (position 1) and a second position (position 2) as if both vectors extend from a common reference point or origin. Typically, position 1 is selected to correspond to the vector normal at the start of a limb movement and position 2 is the vector normal at the end of that limb movement. In this configuration, the ROM calculation algorithm calculates an angle that corresponds to angular rotation of the limb about its joint between the start and end positions of the limb movement. As previously mentioned, the clinician may use the measurement device to perform an assessment of a limb movement in a movement plane having any orientation in 3D space, including vertical or horizontal planes, or any arbitrary plane. The measurement device is also optionally configured to calculate information indicative of the orientation of the movement plane through which the limb was moved during an assessment as further explained below. Therefore, the measurement device can be configured to provide the user with both information on the angular rotation data (e.g. ROM angle) for a limb movement along with information indicative of the orientation of the movement plane in 3D space through which the limb was moved.

In this embodiment, the movement plane is defined as the plane extending between or through the vectors normal to the contact pad of the measurement device at positions 1 (e.g. start position) and 2 (e.g. end position) of the limb movement as if the vectors extend from a common reference point.

A vector normal to the movement plane V_{N} can be determined by finding the cross-product of V_{z1} and V_{z2} as follows:

$$\{ \begin{matrix}{V}_{xN} \\ {V}_{yN} \\ {V}_{zN}\end{matrix} \} = \{ \begin{matrix}{N}_{z 1} \\ {E}_{z 1} \\ {A}_{z 1}\end{matrix} \} \times \{ \begin{matrix}{N}_{z 2} \\ {E}_{z 2} \\ {A}_{z 2}\end{matrix} \} \qquad ( 8 )$$

V_{N} represents a vector normal to the movement plane and this information can be processed and fed back to the clinician in various forms. For example, the orientation of the movement plane as defined by V_{N} can be fed back or displayed to the user on the measurement device textually, numerically, graphically, or otherwise. In one embodiment, the V_{N} information may be processed to generate a movement plane orientation angle for feeding back or displaying to the clinician. For example, the movement plane orientation angle could be calculated relative to a reference plane, for example, either a horizontal plane or a vertical plane. More particularly, the movement plane orientation angle may be calculated to represent the difference in angle (on the acute side) between movement plane and reference plane about the line of intersection between the planes. In another embodiment, a movement plane may be graphically represented and displayed to the user with reference to a global reference frame. In yet another embodiment, the orientation of the movement plane may be textually represented as being either ‘horizontal’ or ‘vertical’ if oriented as such within preset tolerance ranges or alternatively arbitrary.

### Horizontal and Vertical Limb Movement Assessments and Feedback Algorithm

Although clinicians may perform limb assessments through a movement plane in any arbitrary orientation, many of the standardised limb movements have been confined to vertical or horizontal planes. In one embodiment, the measurement device may be configured to feedback information to the clinician about whether the orientation of the movement plane corresponds to either a horizontal plane or a vertical plane relative to predetermined tolerance ranges for each plane. For example, at the end of a limb movement assessment the control system of the measurement device is configured to determine using the information from equation (8) whether the orientation of the movement plane is in either a horizontal plane or a vertical plane and may feed this information back to the clinician either audibly, via the display, or otherwise. In particular, the measurement device can be configured to warn the clinician if the device and limb of the patient has moved in a movement plane that is not substantially horizontal or vertical within preset or predetermined tolerance ranges of those planes in a global reference frame and that the clinician should disregard the result and perform the limb assessment again until the correct plane of movement is achieved.

In this embodiment, V_{N} is a normalised or unit vector. For a vertical plane of movement, the vertical component of V_{N}, V_{zN}, should be close to 0, and for horizontal plane of movement, V_{zN} should be close to absolute 1. It is generally not possible for a clinician to measure a plane of movement that is perfectly aligned with either vertical or horizontal plane, so as mentioned above, the control system can be configured to check whether the orientation of the movement plane is in a vertical or a horizontal plane within preset tolerance ranges for each plane. By way of example only, the tolerance range for the vertical plane may be set to −0.1 to 0.1 and the tolerance range for the horizontal plane may be set to cover the range −0.9 to −1.0 and 0.9 to 1.0, although it will be appreciated that the tolerance ranges may be varied depending on the accuracy required.

By way of example only, one possible movement plane assessment algorithm for checking whether the orientation of the movement plane is in a horizontal or a vertical plane is as follows, although it will be appreciated that various other algorithms could be used:

if (abs(VzN)<0.1)
then the plane of movement is vertical
   else if (abs(VzN)>0.9)
then the plane of movement is horizontal
   else
warn the user that the plane is neither vertical nor horizontal.

It will be appreciated that information from the movement plane assessment of the above algorithm may be fed back to the user in various ways. For example, the control system may be configured to advise the clinician audibly and/or visually as to whether the movement plane of the assessment was or wasn't in either the horizontal or vertical planes within their respective tolerance ranges. It will also be appreciated that the movement plane assessment algorithms may be simplified into checking for only either horizontal or vertical plane movements based on an input from the clinician indicative of the desired movement plane for assessment.

Typically, it is preferable for both the ROM angle, θ, and the vector normal to the movement plane, V_{N}, to be determined by comparing the 3D orientation data (e.g. rotation matrixes R) at or toward the start and end positions of the limb movement. For example, in one embodiment, the first and the last set of recorded data for the 3D orientation of the device are used for the measures of ROM and the orientation of the corresponding movement plane.

### Typical Operation of the Measurement Device

Clinicians are interested in measuring the range-of-motion (ROM) of various joints in the body. A complete ROM measurement should have three angle values: start ROM, end ROM, and total ROM (which is the difference between end ROM and start ROM). There is a defined anatomical joint reference angle or axis for each particular joint, and the ROM measurement must be referenced from that angle or relative to that axis. However, there are several motions associated with each joint, so an angle measurement should always be accompanied by the direction of the motion.

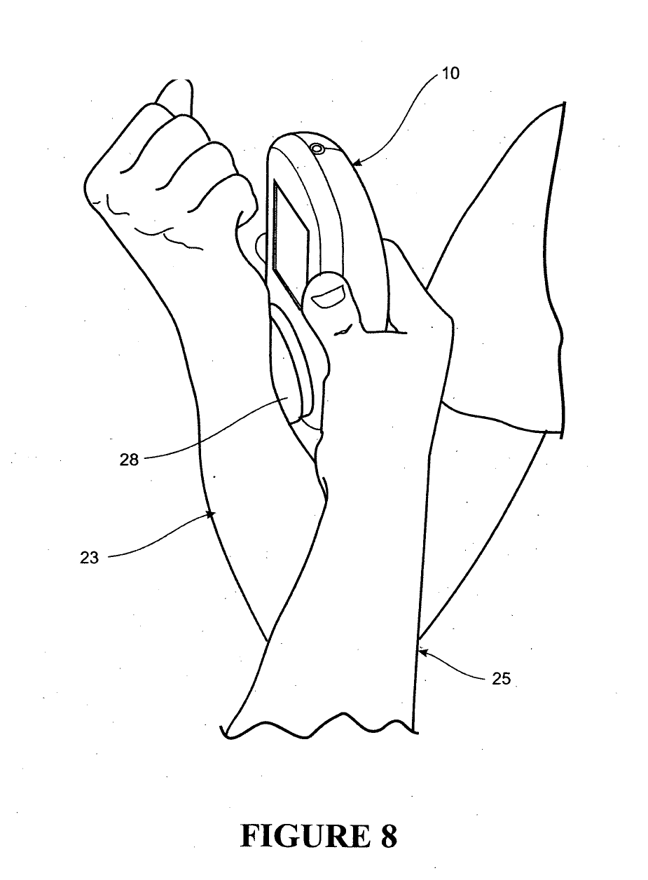

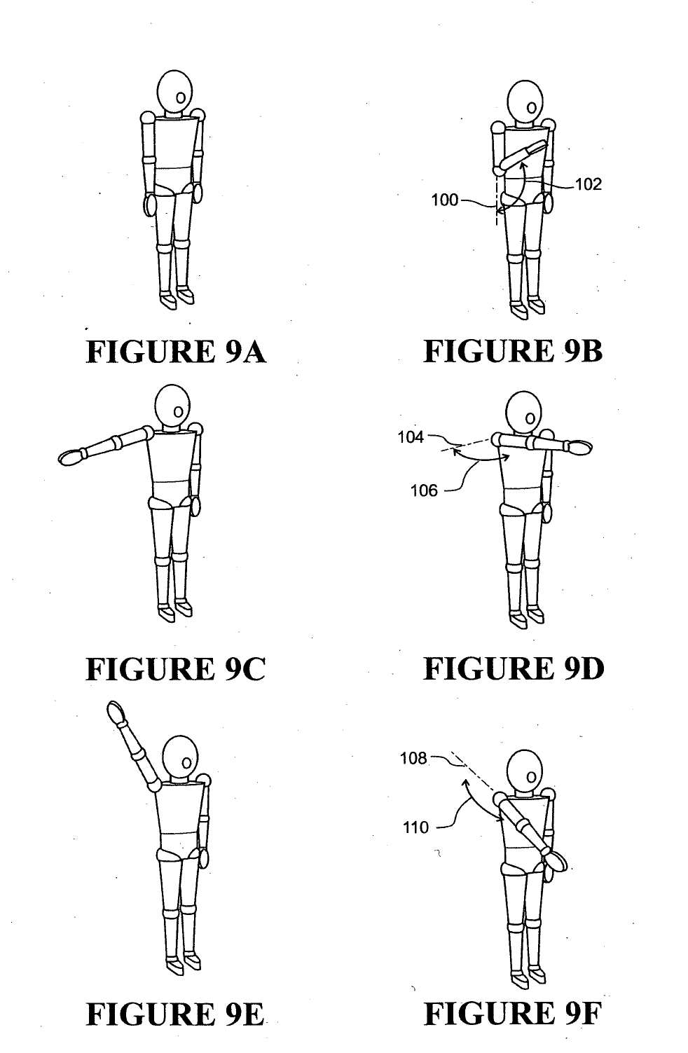

With reference to FIGS. 9a and 9b, a right elbow flexion limb movement is shown. The ROM of a right elbow flexion, is the flexion motion of the right elbow from a straight arm (typically 0° start ROM, but may be as much as −7° hyper-extended or +10° contracted) as shown in FIG. 9a to a bent arm (typically 150° end ROM) as shown in FIG. 9b. The anatomical joint reference axis or anatomical zero for this limb movement is the white line 100 shown when the upper arm is aligned with the forearm in FIG. 9b. The arrow 102 depicts the vertical movement plane for the right elbow flexion.

A more complicated limb movement is shown in FIGS. 9c and 9d, which is the horizontal adduction of the right shoulder. The anatomical joint reference axis or anatomical zero is the white line 104 shown when the upper arm is aligned with the frontal plane as shown in FIG. 9d. The arrow 106 depicts the horizontal movement plane for the adduction of the right shoulder.

FIGS. 9e and 9f show an example of a limb movement of the right shoulder through an arbitrary plane in 3D space having both horizontal and vertical components. Again the white line 108 depicts the anatomical joint reference axis and arrow 110 depicts the arbitrary movement plane through which the limb is moved or rotated about the shoulder joint.

The movement plane of rotation of the measurement device can be in any direction, whether horizontal, vertical or any arbitrary combination of the two. As previously mentioned, the control system may be arranged to restrict ROM measurements to two planes of rotation, such as the vertical and horizontal planes, in a manner previously described by calculating and feeding back to the clinician information relating to the orientation of the movement plane. However, the measurement device is capable of measuring ROM of limb movements through any arbitrary plane in 3D space, along with calculating and displaying information, whether numerical, textual, or graphical, indicative of the orientation of the movement of plane extending between the start and end positions of the ROM angle measurement.

Prior to assessing a particular limb movement with the device, the clinician must set the anatomical joint reference axis into the device. This is done by holding the device against the limb while it is in the anatomical zero position, and then operating a trigger switch 19 to record the 3D orientation of the device in the global coordinate system in that position. In particular, the control system will be activated by operation of the trigger switch to generate a rotation matrix representing the 3D orientation of the device while the limb is in the anatomical zero position. The limb is then moved through the relevant movement plane, for example vertical, horizontal or arbitrary depending on the joint and movement being assessed, and of the rotation limb relative to the anatomical zero is continuously recorded using the algorithms previously described. In addition, the force applied between the limb and contact pad over the ROM is simultaneously recorded from the force sensor signals.

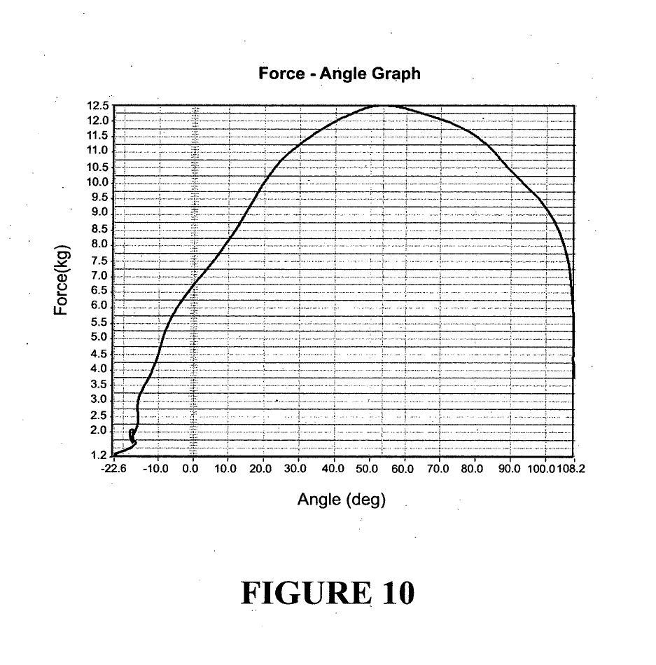

Referring to FIG. 10, a typical graphical result of the data recorded by the measurement device for a right elbow flexion is shown. The graph shows the strength of the joint with respect to its rotation relative to the anatomical zero. In the result, the start ROM is at −22° because the joint is hyper-extended. The end ROM is at 108° so the total ROM is 130°.

### Performing Dynamic Measurements

The measurement device can be used by clinicians or physiotherapists to dynamically measure joint strength over ROM. The clinician presses the contact pad of the device against a limb to resist the patient's movement and then instructs the patient to push against the device and thereby move (e.g. flex or extend) their limb with maximal effort through its full ROM in a particular movement plane. There are two possible modes of resistance: concentric is when the external resistive force opposes the direction of the motion (the patient overpowers the physiotherapist); and eccentric where the resistive force is in the same direction of the motion (the physiotherapist overpowers the patient). In either, the clinician typically applies a resistance force to control limb movement to maintain a constant speed of joint rotation. Naturally, the resisting force should be aligned with the vector normal to the contact pad of the device (e.g. z-axis of the measurement device), and the limb movement should be carried out in approximately 2-dimensional movement plane. As previously mentioned, the orientation of the movement plane can be identified throughout the movement by continuously tracking movement of the z-axis of the measurement device. It will be appreciated that the movement plane, whether horizontal, vertical, or arbitrary, will depend on the joint being assessed and the type of limb movement. Ideally, the clinician instructs the patient to confine their limb movement to the movement plane of interest.

The function of the measurement device is to measure muscle strength (force) and joint motion (angle) simultaneously. This is achieved by placing the contact pad of the measurement device at an appropriate location on the limb to be measured. In order for the device (being held by the clinician) to resist the force and motion of the limb, the contact pad should preferably be perpendicular to the direction of the patient's force as previously mentioned. Using this principle, the ROM measurement is the angle that the contact pad rotates during the limb movement, as previously described.

The measurement device can be used as a handheld dynamometer in that it can be arranged to calculate the mechanical energy or power of a limb moving through a ROM. In particular, the device can measure the energy dissipated (or work done) by a limb. From a previous publication on isokinetic dynamometers [Baltzopoulos & Brodie 1989], it has been established that if the speed of the motion is less than 60 degrees per second (°/s), the force generated by the limb remains constant. Based on this information, the clinician should preferably resist the motion of a limb so that the speed of motion though the range does not exceed 60°/s. Taking this approach, the energy measurement by the measurement device should be maximal and similar to that measured by an isokinetic dynamometer system at speed not greater than 60°/s. It will be appreciated that the device can be arranged to calculate the angular velocity of the limb movement from the angular rotation data, and therefore can be arranged to warn the clinician if the maximum angular velocity is exceeded.

To record the force applied by the limb through its ROM, multiple measurements of the 3D orientation and force sensors are required. Before a limb movement is measured, the anatomical joint reference axis (zero reference) must be first established by aligning the device against the limb while it is at the anatomical zero position as previously described. The vector normal to the force plate at this location, Vₒ, is stored when a trigger switch or button 19 is actuated on the measurement device by the clinician. The clinician then actuates the trigger a second time to start recording the 3D orientation of vector V_{Z}, from the data generated by the 3D orientation sensor and the force, Fᵢ, from the data generated by the force sensor. The reference i represents each successive sample of 3D orientation data and force data sensed during a limb movement where i=0, 1, 2, 3 . . . n, and where i=0 is the start position of the limb movement and i=n is the end position of the limb movement. It will be appreciated that the total number of data sets generated will depend on the sample rate of sampling of the sensors and time taken to conduct the limb movement from start to finish and these factors can be varied as desired. The 3D orientation of the vector is extracted from the series of rotation matrices (Rᵢ) that are generated during the limb movement from the signals of the 3D orientation sensor. The recording stops when the trigger is clicked again. The i^{th} ROM angle can be calculated using equation (7) between V_{z0} and V_{zi}. The patient's strength, Fᵢ, can be recorded as force (N) against the contact pad or, if the therapist wishes, the limb torque can be calculated by multiplying Fᵢ by the moment arm distance, that is, the distance from the centre of the contact pad to the centre of the joint. It will also be appreciated that the orientation of the movement plane between V_{z0} and each V_{zi} can be calculated with equation (8), although typically the orientation of the movement plane is defined between the start and end of the limb movement, i.e. between V_{z0} and V_{zn}.

The work done by the patient can be calculated by integrating the torque versus ROM angle measurements. If the unit for torque is the Newton-metre (Nm), and the unit for the angle is the Radian (Radian=Degree×π/180), then the area under the torque versus angle graph gives energy dissipated (work done) in Joules (J). It will be appreciated that the time taken to complete a limb movement from start to finish can be recorded by a timing module to enable the average speed of the limb movement to be calculated.

### Output Display and Data Storage

The data recorded by the measurement device can be stored in onboard memory or transferred to Personal Computer as described. The user interface of the measurement device may be operated by the clinician to display measured data from a limb movement in various forms, including numerically displaying strength (peak, maximum, average for example), start ROM, end ROM, total ROM, speed of limb movement in ⁰/s, information indicative of the orientation of the movement of plane and the like. The user interface may also be arranged to plot and display a graph of strength (force) over ROM (angle) for a limb movement. Historical data for a particular patient can also be retrieved from memory and compared against current measurements to gauge process of the patient.

### Alternative 3D Orientation Processing Algorithms

In the first preferred form, the rotation matrix method is employed to store, process and extract the necessary device 3D orientation information for calculating the angular rotation data and ROM. In alternative forms, the 3D orientation of the device with respect to a fixed global coordinate system can be represented in other ways if desired, such as using the three Euler angles (pitch, yaw, and roll) or using the quarternion method, where an orientation can be defined with one rotation angle and a vector (x, y, z components) defining the axis of rotation. The rotation from one quarternion (one orientation) to another can be found using a quarternion multiplication operation.

### Second Preferred Form—Handheld Sensor Unit

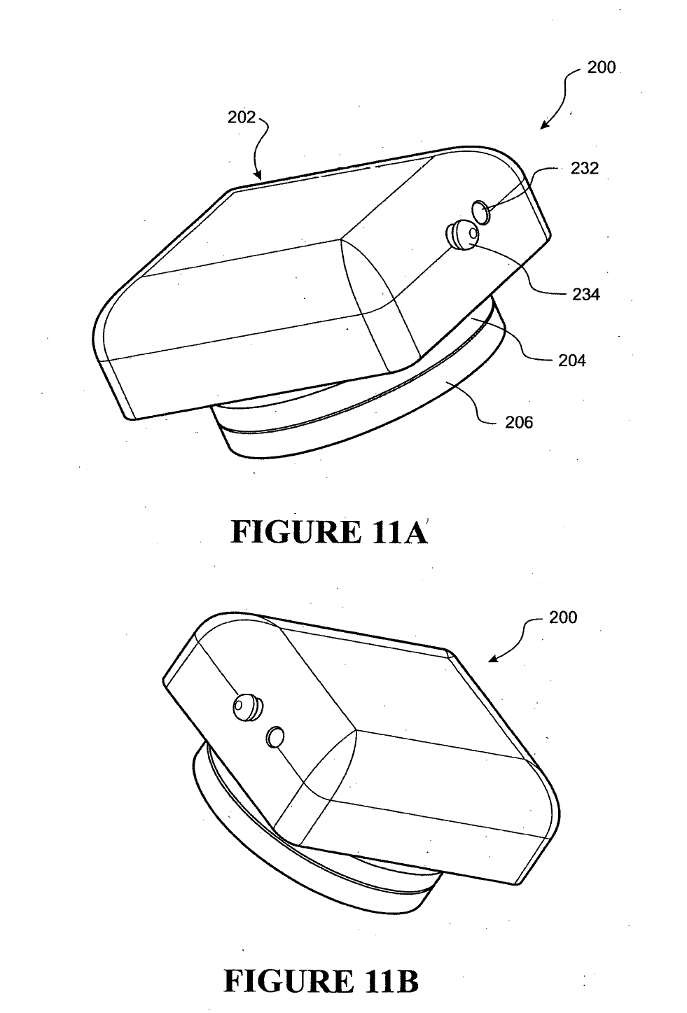

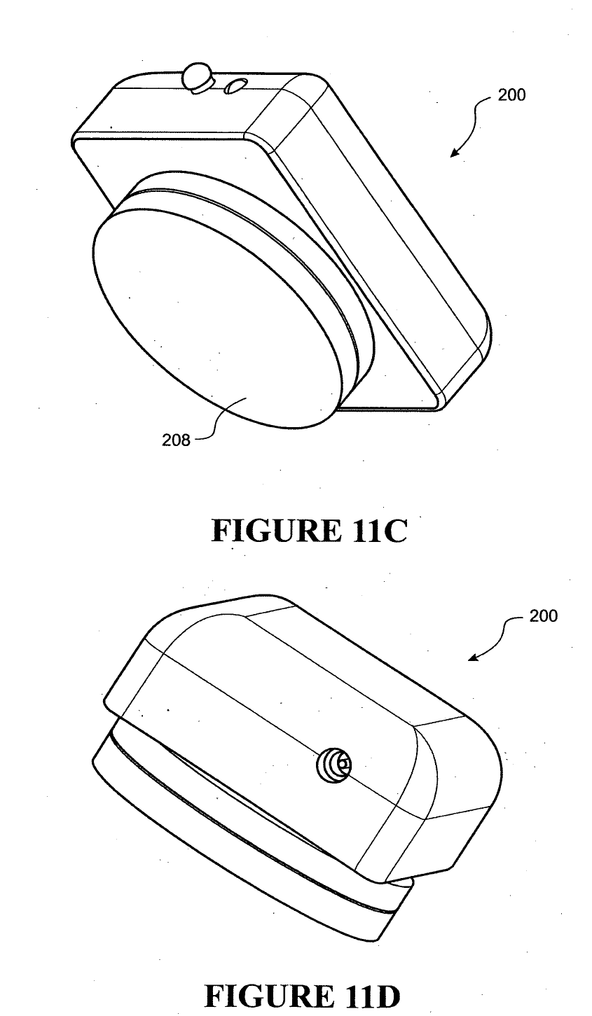

FIGS. 11a-11d show a second preferred form of the measurement device in the form of a handheld sensor unit 200. The handheld sensor unit 200 is similar to the first preferred form handheld measurement device 10, but excludes the processing and display functionalities. In particular, the handheld sensor unit 200 comprises a handheld housing 202 within which the 3D orientation sensor and force sensor are provided. As with the first preferred form, the force sensor comprises a force transducer 204 that is coupled to a contact pad or plate 206. The contact pad 206 provides the contact surface 208 for contacting a part of the limb during a limb measurement.

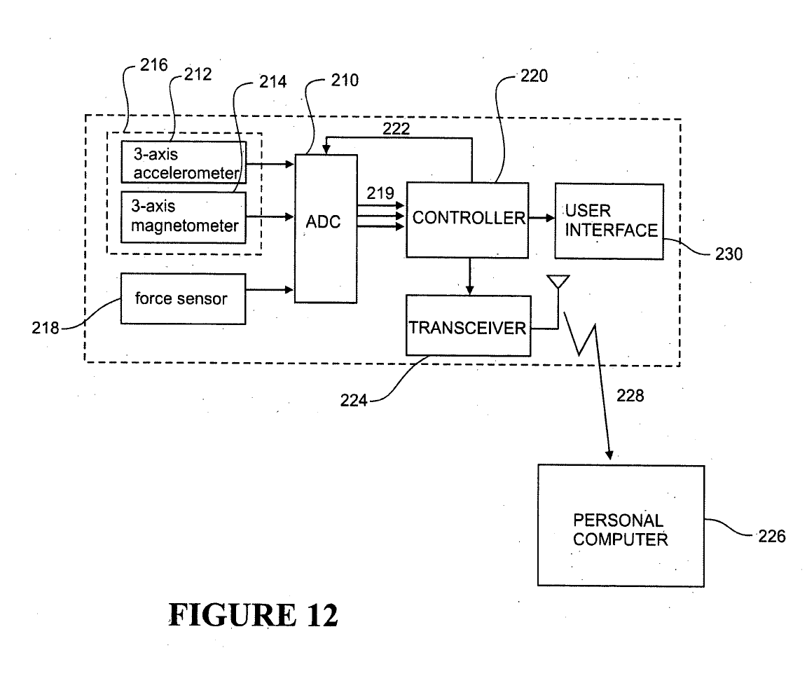

With reference to FIG. 12, the main modules of the handheld sensor unit are shown. In brief, the control system onboard the handheld sensor unit is arranged to receive 3D orientation sensor and force sensor signals and transmits those signals wirelessly to an external device, such as a Personal Computer, via a radio link. In the second preferred form, an analogue-to-digital (ADC) converter 210 receives the voltage signals from the 3-axis accelerometer 212 and 3-axis magnetometer 214 of the 3D orientation sensor 216, and a voltage signal from the force sensor 218, and converts these into respective digital signals 219. As with the first preferred form control system, a main controller 220 operates the ADC 210 via a control signal 222 and receives the digital signals 219. The 3D orientation and force data signals 219 are received concurrently by the controller 220 and this then sends the data to a transceiver module 224. The transceiver module 224 is arranged to continuously transmit the measured raw data from the sensors 216,218 in real-time to an external device, such as a Personal Computer 226, via a radio link 228. It will be appreciated that any wireless communication protocol could be used, including WiFi, Bluetooth and the like. Alternatively, an infrared communication link could be used to transmit the data. It will be appreciated that the Personal. Computer may have a wireless transceiver module onboard that is configured to receive the data from the handheld sensor unit or may be connected to a stand-alone customised transceiver module that is configured to communicate with the sensor unit.

In the second preferred form, a user interface 230 is provided on the sensor unit. With reference to FIG. 11a, the user interface may comprise a LED 232 or similar for indicating power status, and a trigger, switch or button 234 for starting and stopping the recording/transmission of data from the sensors. The button 234 may also be configured to set the anatomical joint reference axis prior to a limb movement measurement as previously described in relation to the first preferred form device.

The operation of the handheld sensor unit 200 by the clinician is the same as that for the first preferred form handheld measurement device. The only difference being that the handheld sensor unit does not process, display or store the measurement results as is possible with the handheld measurement device. The data processing functions are carried out on the Personal Computer using the algorithms described above.

It will be appreciated that the algorithms previously described may be implemented in standalone customised hardware or alternatively in software running on general purpose computers or other computing devices with memory. It will be appreciated that the algorithms and processing steps may be encoded in software as a set of computer readable instructions for execution by a computer. The computer readable instructions may be stored in any form of memory or alternatively in any computer readable storage medium, whether magnetic, optical, solid-state, or otherwise.

### Database Management System

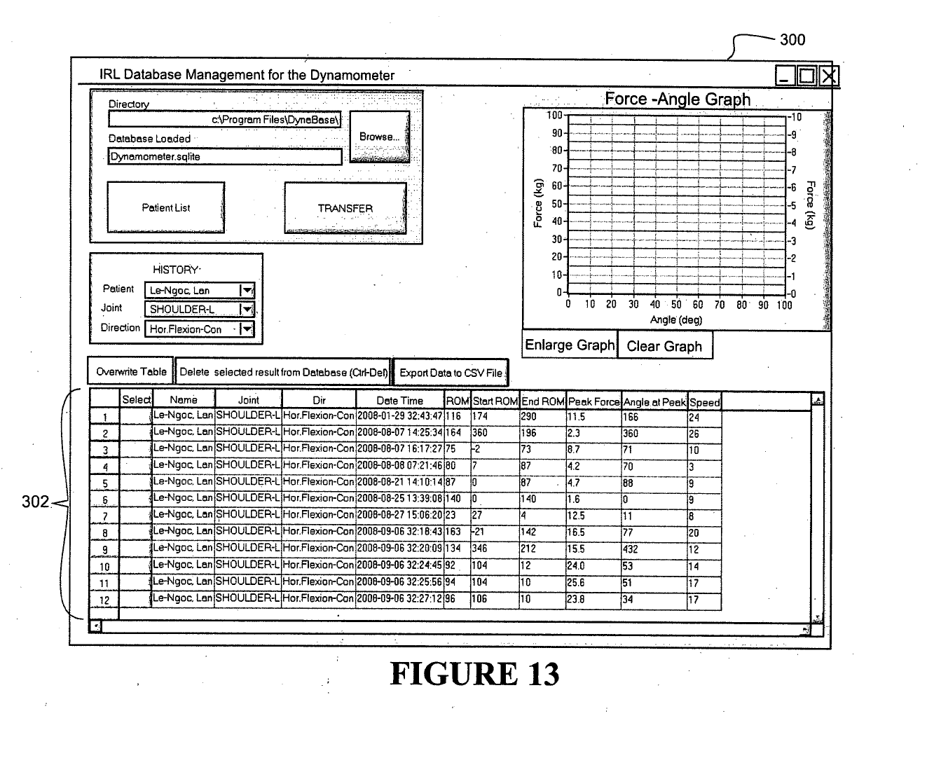

The data obtained from the handheld measurement device or sensor unit can be transferred to a Personal Computer or similar for storage and processing if desired. The Personal Computer may be provided with software for managing and manipulating the data. FIG. 13 shows a possible user interface 300 for the data management system running on the Personal Computer. The user interface 300 is arranged to display historical measured data 302 for a patient for particular joint and limb movements. Each set of data relates to a particular joint (for example left shoulder) and a limb movement type (for example horizontal rotation). Each different limb movement type may have an associated movement plane and direction of rotation in that plane, along with a predetermined anatomical joint reference axis. The patient data can include the date and time of measurements, total ROM, start ROM, end ROM, peak force during the limb movement, angle at peak force, average speed, information indicative of the orientation of the movement plane between the start and end positions of the limb movement and the like.

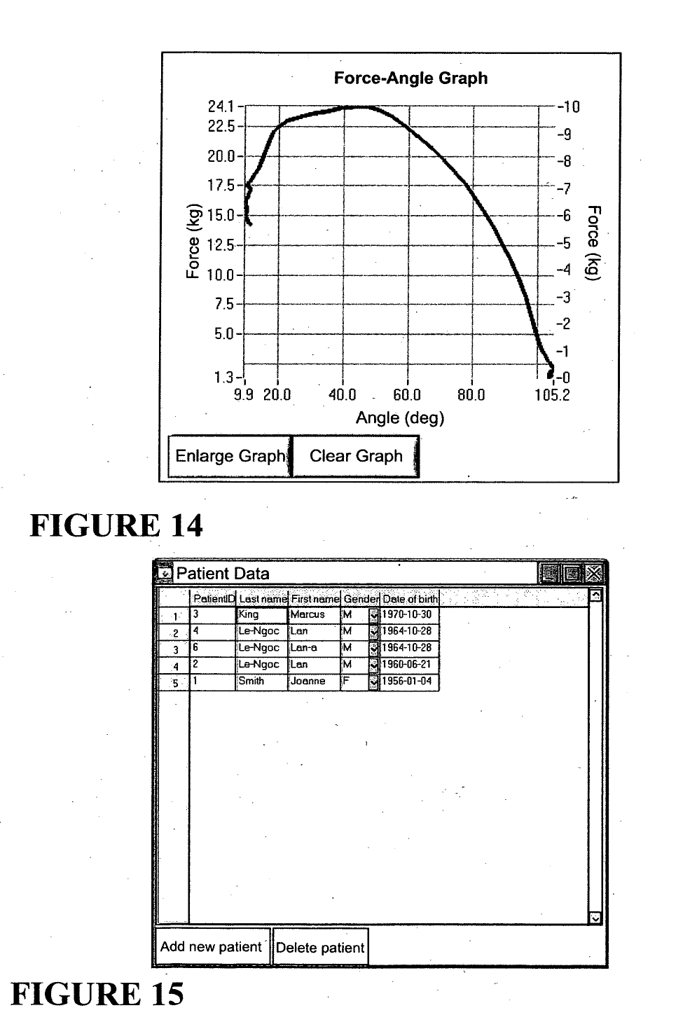

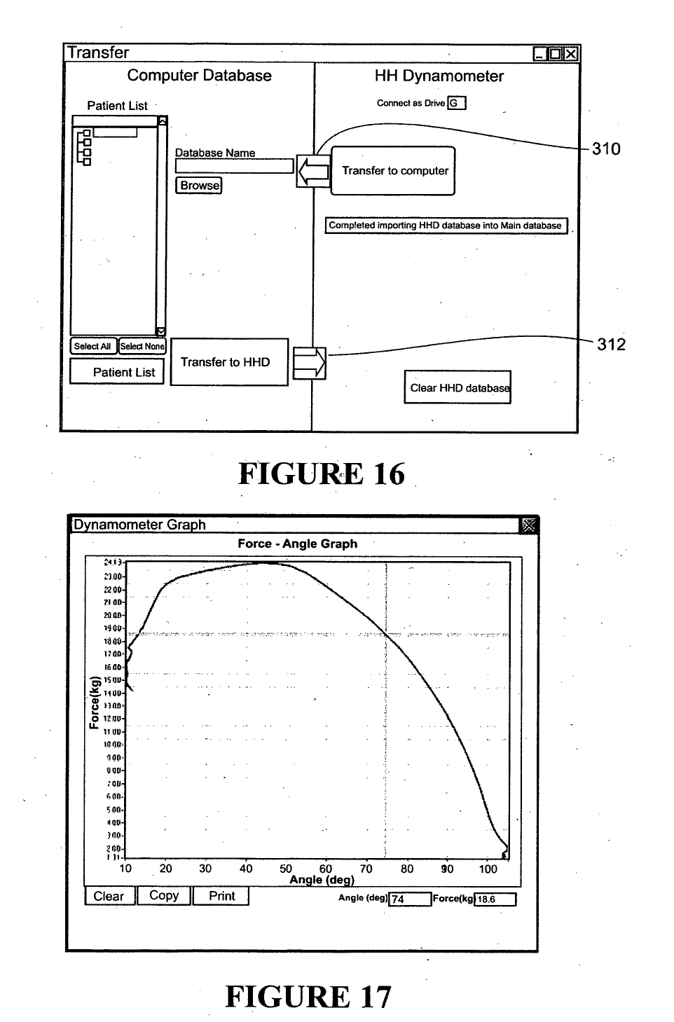

FIGS. 14 and 17 show force versus angle graphs that can be plotted from the patient measurement data. It will be appreciated that comparisons of the patients measured data over time, either numerically or graphically, can be made with the data management software. FIG. 15 shows a screenshot of a preferred patient data module of the database management system in which the clinician may enter patient data, including, for example, a patient ID, name, gender, date of birth or any other relevant information. FIG. 16 shows a screenshot of a preferred data transfer module for transferring data between the measurement device and database management system. The data transfer module enables patient data to be uploaded 310 from the measurement device to the Personal Computer or downloaded 312 from the Personal Computer to the measurement device, either wirelessly or via a hardwired connection as described previously.

### Advantages and Benefits of Measurement Device

A primary benefit of the measurement device and sensor unit is that it comprises a 3D orientation sensor that can measure rotation of a limb about a joint in any arbitrary plane, whether horizontal, vertical or a combination of both. This provides significant advantages in that the clinician can measure a patient's limb movement regardless of their position or orientation, including whether they are lying, sitting, or standing for example.

As mentioned, the measurement device and sensor unit can measure range of motion of a joint in any arbitrary plane in 3D space and with reference to an anatomical joint reference axis set by the clinician before the limb exercise. The measurement device is capable of measuring hinge-type joints such as elbows and knees, whether the patient is standing, lying or sitting down, and also ball-type joints such as hips and more complex joints such as shoulders and ankles.

In the preferred form, the measurement axis of the dynamometer is the vector normal to the contact surface of the force sensor, which the patient is asked to press against. The control system associated with the measurement device is arranged to process absolute 3D orientation information relating to the device and convert it to a measure of ROM of a limb movement about a joint in any direction of a movement plane. More particularly, the measurement device can generate a continuous measurement of the angle of rotation of the limb about a joint through any arbitrary plane of movement with reference to a user set anatomical reference axis (or zeroed axis) in that plane. In addition, the measurement device is then able to present a single ROM angle to the clinician for assessment. The measurement device can measure and track the force applied to the force sensor against the angle of rotation of the limb about the joint. Additionally, the measurement device can measure and output information indicative of the orientation of the movement plane through which the limb was moved.

The foregoing description of the invention includes preferred forms thereof. Modifications may be made thereto without departing from the scope of the invention as defined by the accompanying claims.

1. A handheld measurement device for enabling a user to measure a person's muscle strength and range of motion associated with a limb movement about a joint in a movement plane, comprising:
a handheld housing having a contact surface that is arranged to contact a part of the person's limb during the limb movement;
a 3D orientation sensor mounted within the housing that is arranged to sense the 3D orientation of the device in 3D space and generate representative 3D orientation signals during the limb movement;
a force sensor associated with the contact surface that is arranged to sense the force applied by the person's limb to the contact surface and generate representative force signals during the limb movement; and
a control system that is arranged to concurrently receive the 3D orientation signals and force signals from the respective sensors during a limb movement and process those signals to generate force data indicative of the force applied by the person's limb to contact surface during the limb movement and angular rotation data indicative of the angle of rotation of the limb about the joint in the movement plane during the limb movement.

2. A handheld measurement device according to claim 1 wherein the control system is arranged to process the 3D orientation signals from the 3D orientation sensor to generate 3D orientation representations of the device with reference to a 3-axis local device coordinate system and a 3-axis global coordinate system during the limb movement.

3. A handheld measurement device according to claim 2 wherein the 3D orientation sensor comprises a 3-axis accelerometer that is arranged to generate accelerometer signals representing the three orthogonal components of the gravity vector in the local device coordinate system and a 3-axis magnetometer that is arranged to generate magnetometer signals representing the three orthogonal components of the Earth's magnetic field vector in the local device coordinate system, and wherein the control system is arranged to generate the 3D orientation representations based on the accelerometer and magnetometer signals.

4. A handheld measurement device according to claim 2 or claim 3 wherein the control system is arranged to generate the angular rotation data based on the orientation of a reference vector in the local device coordinate system.

5. A handheld measurement device according to claim 4 wherein the control system is arranged to extract the orientation of the reference vector from the 3D orientation representations of the device during the limb movement.

6. A handheld measurement device according to claim 4 wherein the reference vector is a vector substantially normal to the contact surface of the handheld housing.

7. A handheld measurement device according to claim 4 wherein the angular rotation data represents the angular rotation of the reference vector in the movement plane and which corresponds to the angular rotation of the limb about its joint in the movement plane.

8. A handheld measurement device according to claim 4 wherein the control system is arranged to generate angular rotation data in the form of a single Range of Motion (ROM) angle representing the angle between the reference vector at the start and end of a limb movement based on a dot-product calculation of the start and end reference vectors.

9. A handheld measurement device according to claim 4 wherein the control system is further arranged to extract from the 3D orientation representations information indicative of the orientation of the movement plane for a limb movement relative to the 3-axis global coordinate system.

10. A handheld measurement device according to claim 9 wherein the movement plane is defined as the plane extending between the reference vectors at the start and end positions of a limb movement.

11. A handheld measurement device according to claim 9 wherein the control system is arranged to output information indicative of whether the orientation of the movement plane corresponds to a substantially horizontal plane in the global coordinate system within a predefined tolerance range.

12. A handheld measurement device according to claim 9 wherein the control system is arranged to output information indicative of whether the orientation of movement plane corresponds to a substantially vertical plane in the global coordinate system within a predetermined tolerance range.

13. A handheld measurement device according to claim 9 wherein the control system is arranged to generate a movement plane orientation angle representing the orientation of the movement plane relative to a reference plane.

14. A handheld measurement device according to claim 9 wherein the control system is arranged to generate information indicative of the orientation of the movement plane by determining the vector normal to the movement plane based on a cross-product calculation of the start and end reference vectors.

15. A handheld measurement device according to claim 2 wherein the control system is arranged to generate the angular rotation data representing the angular rotation of the limb about the joint relative to a preset anatomical joint reference axis.

16. A handheld measurement device according to claim 15 wherein the control system is operable to extract the anatomical join reference axis from the 3D orientation representation of the device when the limb is in contact with the contact surface of the device and aligned with the desired anatomical joint reference axis.

17. A handheld measurement device according to claim 15 wherein the control system further comprises a user interface that is operable by a user to set and store the anatomical joint reference axis prior to a limb movement measurement.

18. A handheld measurement device according to claim 2 wherein the control system is arranged to generate the 3D orientation representations of the device in the form of 3×3 rotation matrices comprising values that represent the absolute orientation of this device in the global coordinate system.

19. A handheld measurement device according to claim 1 wherein the control system further comprises a user interface and is arranged to receive input from a user via the user interface as to the start and end positions of a limb movement and wherein the control system is arranged to generate angular rotation data in the form of a ROM angle of the limb movement between the start and end positions.

20. A handheld measurement device according to claim 19 wherein the control system is arranged to generate the ROM angle based on the total angular rotation of a vector normal to the contact surface between the start and end positions of the limb movement in the movement plane.

21. A handheld measurement device according to claim 1 wherein the control system is arranged to generate force data and angular rotation data representing the force applied by the limb to the contact surface and the corresponding angular position of limb during the limb movement so as to generate measurement data indicative of muscle strength over the entire ROM of the limb movement.

22. A handheld measurement device according to claim 1 wherein the control system is arranged to generate force data comprising any one of the following: peak force, maximum force, or average force strength based on the force applied over the entire limb movement.

23. (canceled)

24. A handheld measurement device according to claim 1 wherein the 3D orientation sensor comprises one or more accelerometers and one or more gyroscopes that are together arranged to sense the 3D orientation of the device in 3D space and generate representative 3D orientation signals.

25. (canceled)

26. (canceled)

27. A handheld measurement device according to claim 1 wherein the movement plane may be any of the following: horizontal, vertical or arbitrary.

28. A handheld sensor unit for enabling a user to measure a person's muscle strength and range of motion associated with a limb movement about a joint in a movement plane, comprising:
a handheld housing having a contact surface that is arranged to contact a part of the person's limb during the limb movement;
a 3D orientation sensor mounted within the housing that is arranged to sense the 3D orientation of the device in 3D space and generate representative 3D orientation signals during the limb movement;
a force sensor associated with the contact surface that is arranged to sense the force applied by the person's limb to the contact surface and generate representative force signals; and
a control system that is arranged to concurrently receive the 3D orientation signals and force signals from the respective sensors during a limb movement and transmit those to an external device.

29. A handheld sensor unit according to claim 28 wherein the control system comprises a communication module that is arranged to transmit the 3D orientation signals and force signals to an external device; and
wherein the communication module is configured for wired connection and transmission of data with an external device.

30. (canceled)

31. A handheld sensor unit according to claim 28 wherein the control system comprises a communication module that is arranged to transmit the 3D orientation signals and force signals to an external device; and
wherein the communication module is configured for wireless communication of data with an external device.

32. A handheld sensor unit according to claim 28 wherein the control system further comprises a user interface to enable a user to operate the sensor unit to begin sensing at the start position of the limb movement and halt sensing at the end position of the limb movement.

33. A method of measuring a person's muscle strength and range of motion
associated with a limb movement about a joint, comprising the steps of:
(a) applying the contact surface of a handheld measurement device or sensor unit of claim 1 to a part of the person's limb with resistance;
(b) causing the person to move their limb through its full range of motion about the joint in a movement plane;
(c) measuring the force signals and 3D orientation signals from the sensors of the device or unit during the limb movement; and
(d) processing the force signals and 3D orientation signals to generate output data representing the person's muscle strength over their range of motion for the limb movement.

34. A method according to claim 33 wherein step (d) comprises generating 3D orientation representations of the device or unit with reference to a 3-axis local device coordinate system and a 3-axis global coordinate system based on the 3D orientation signals.

35. A method according to claim 34 wherein step (d) comprises generating the 3D orientation representations of the device or unit in the form of rotation matrices that represent the absolute orientation of the device or unit in the global coordinate system; and processing the series of rotation matrices to generate angular rotation data representing the angle of rotation of the limb about the joint based on the rotation of a reference vector in the local device coordinate system.

36. (canceled)

37. A method according to claim 35 wherein the reference vector is substantially
normal to the contact surface of the device or unit.

38. A method according claim 35 wherein step (d) comprises generating a measurement of range of motion of the limb based on the total angle of rotation of the reference vector in the movement plane between the start and end positions of the limb movement.

39. A method according to claim 33 wherein the method further comprises the step of setting an anatomical joint reference axis prior to starting the limb movement by aligning the person's limb within the desired anatomical joint reference axis and operating the device or unit to extract and store the anatomical joint reference axis based on the 3D orientation signals sensed at that position; and wherein angular rotation data representing the angle of rotation of the limb about the joint is generated relative to the stored anatomical joint reference axis.

40. A method according to claim 33 wherein the 3D orientation sensor comprises an accelerometer and a magnetometer, and wherein step (d) comprising the steps of:
(e) defining a 3-axis local coordinate system for the sensor and a 3-axis global coordinate system;
(f) receiving accelerometer and magnetometer signals during the limb movement;
(g) generating rotation matrices representing the absolute 3D orientation of the sensor with reference to the 3-axis local coordinate system and 3-axis global coordinate system;
(h) processing the rotation matrices to extract angular rotation data relating to the angular rotation of a reference vector of the 3-axis local coordinate system in the movement plane of the 3-axis global coordinate system; and
(i) generating a measurement of angular rotation of the limb based on the angular rotation data.

41. A method according to claim 40 wherein step (i) comprises generating a measurement of the total angular rotation of the limb during the limb movement.

42. A method according to claim 40 wherein step (i) comprises generating a measurement of the angular rotation of the limb with reference to an anatomical joint reference axis.

43. A method according to claim 42 wherein the method further comprises the step of setting an anatomical joint reference axis prior to limb movement.

44. A method of measuring a person's muscle strength and range of motion associated with a limb movement about a joint, comprising the steps of:
(a) applying the contact surface of a handheld measurement device or sensor unit of claim 28 to a part of the person's limb with resistance;
(b) causing the person to move their limb through its full range of motion about the joint in a movement plane;
(c) measuring the force signals and 3D orientation signals from the sensors of the device or unit during the limb movement; and
(d) processing the force signals and 3D orientation signals to generate output data representing the person's muscle strength over their range of motion for the limb movement.
